(19) United States
(12) Patent Application Publication
(10) Pub. No.: US 2014/0370510 A1
(43) Pub. Date: Dec. 18, 2014

(54) MODULATION OF PEPTIDOME USING HISTONE DEACETYLASE INHIBITOR

(71) Applicants: George Blanck, Tampa, FL (US); Kevin Jeremiah Cronin, Tampa, FL (US)

(72) Inventors: George Blanck, Tampa, FL (US); Kevin Jeremiah Cronin, Tampa, FL (US)

(73) Assignee: University of South Florida (A Florida Non-Profit Corporation), Tampa, FL (US)

(21) Appl. No.: 14/361,819

(22) PCT Filed: Feb. 20, 2013
(86) PCT No.: PCT/US2013/026769

§ 371 Date: May 30, 2014

Related U.S. Application Data
(60) Provisional application No. 61/601,845, filed on Feb. 22, 2012.

(51) Int. Cl. G01N 33/50

(52) U.S. Cl. CPC G01N 33/5011; G01N 33/5047; G01N 33/5052
USPC 435/6.12; 435/29

(57) ABSTRACT

Provided herein is a method of modulating an APC peptidome comprising identifying an APC or an APC cell population that expresses an HDACi-associated polypeptide and administering an HDACi to the APC. Also provided herein is a method of identifying an HDACi-associated peptide comprising administering an HDACi to an APC, isolating the polypeptide from an APC MHC molecule binding cleft, and identifying the polypeptide.

### CROSS REFERENCE TO RELATED APPLICATIONS

This application claims the priority benefit of U.S. provisional patent application 61/601,845 filed on Feb. 22, 2012, in the United States Patent Office.

### BACKGROUND

Histone deacetylases (HDACs) are a class of enzymes that remove acetyl groups from an ε-N-acetyl lysine amino acid on a histone. Histones are an important regulator of DNA transcription that are regulated by a cycle of acetylation and deacetylation. Deacetylation prevents transcription of DNA whereas acetylation increases DNA transcription. Deacetylation prevents transcription by increasing the positive charge of the histone tails, thereby encouraging high-affinity binding between the histones and DNA backbone.

Histone deacetylase inhibitors (HDACi's) are a class of compounds that interfere with the action of histone deacetylases. HDACi's have long been used in psychiatry and neurology as mood stabilizers and anti-epileptics. More recently, HDACi's have been used in the treatment of cancer. Suberoylanilide hydroxamic acid (SAHA), trade name Vorinostat, was the first HDACi approved by the FDA for cancer treatment, specifically for cutaneous T Cell lymphoma. SAHA is currently undergoing numerous clinical trials to test efficacy in other cancers. A second HDACi was later approved for antitumor therapy, and there are currently over a dozen HDACi's in clinical trials for treatment of a wide range of carcinomas.

HDACi's have been used in the treatment of cancer due to their ability to induce cell cycle arrest and apoptosis in tumor cells. Specifically, they lead to hyperacetylation of the nucleosome core of histone proteins by inhibiting HDACs from removing acetyl groups. Acetylated histones allow for gene transcription, and when treated with HDACi's, most of the chromatin becomes hyperacetylated. This hyperacetylated histone state leads to transcriptional activation of a number of genes but more importantly a repression of transcription for the majority of genes, which leads to the cell cycle arrest and apoptosis.

Despite the advances made with HDACi's in recent years, there has been no use of HDACi's to modify the peptidome of an antigen presenting cell. A peptidome is the group of antigens/peptides presented on the surface of a cell with a major histocompatibility complex. Accordingly, there is a need for HDACi compositions and methods for the modification of an antigen presenting cell peptidome.

### BRIEF DESCRIPTION OF THE DRAWINGS

FIG. 1 is a graph that illustrates the alterations in CLIP/HLA-DR ratios in 1286 melanoma cells transformed with CIITA after treatment with DMSO, Entinostat, Vorinostat and IFN-γ.

FIG. 2 is a graph that illustrates the alterations in CLIP/HLA-DR ratios in WM9 melanoma cells transformed with CIITA after treatment with DMSO, Entinostat, Vorinostat and IFN-γ.

FIG. 3 is a graph that illustrates the alterations in CLIP/HLA-DR ratios in Raji B cells transformed with CIITA after treatment with DMSO, Entinostat, Vorinostat and IFN-γ.

FIG. 4 is a graph that illustrates the induction of Cathepsin L1 mRNA by Entinostat in WM9 melanoma cells (FIG. 4A) and the induction of Cathepsin L1 mRNA by Entinostat in Raji B cells (FIG. 4B).

FIG. 5 is a graph that illustrates increased expression of CIITA in cells treated with Entinostat as compared with DMSO.

### DETAILED DESCRIPTION

Provided herein are compositions and methods for modulating a peptidome in an antigen presenting cell (APC). A peptidome is defined herein as a group of peptides presented by, or expressed with, a major histocompatibility complex (MHC) molecule on the surface of an APC under a given set of conditions. A peptidome can be modulated by increasing the amount of one or more polypeptides in the group, decreasing the amount of one or more polypeptides in the group, removing one or more polypeptides from the group, and/or adding one or more polypeptides from the group. Modulation of a peptidome results in a change in the epitope density of a particular APC or APC cell population.

It is a surprising discovery of the present invention that HDACi compositions modulate the peptidome of APCs. Accordingly, provided herein is a method of modulating an APC peptidome comprising identifying an APC or an APC cell population that expresses an HDACi-associated polypeptide and administering an HDACi to the APC. Also provided herein is a method of identifying an HDACi-associated peptide comprising administering an HDACi to an APC, isolating the polypeptide from an APC MHC molecule binding cleft, and identifying the polypeptide.

Definitions

Terms used throughout this application are to be construed with ordinary and typical meaning to those of ordinary skill in the art. However, Applicants desire that the following terms be given the particular definition as defined below.

As used in the specification and claims, the singular form “a,” “an,” and “the” include plural references unless the context clearly dictates otherwise. For example, the term “a cell” includes a plurality of cells, including mixtures thereof.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “antibody” is used in the broadest sense and specifically covers monoclonal antibodies (including full-length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (V_{H}) followed by a number of constant domains. Each light chain has a variable domain at one end (V_{L}) and a constant domain at its other end.

The term “antibody fragment” refers to a portion of a full-length antibody, generally the target binding or variable region. Examples of antibody fragments include Fab, Fab′, F(ab′)₂ and Fv fragments. The phrase “functional fragment or analog” of an antibody is a compound having qualitative biological activity in common with a full-length antibody. For example, a functional fragment or analog of an anti-IgE antibody is one which can bind to an IgE immunoglobulin in such a manner so as to prevent or substantially reduce the ability of such a molecule from having the ability to bind to the high affinity receptor, FeεRI. As used herein, “functional fragment” with respect to antibodies refers to Fv, F(ab) and F(ab′)₂ fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind to a target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains, which enables the sFv to form the desired structure for target binding.

The term “antigen presenting cell” is also referred to as “APC” and is defined herein as a cell that presents or contains a Class II major histocompatibility complex (MHC Class II) at its surface. The term “antigen presenting cell” includes a B cell, dendritic cell, macrophage, activated epithelial cell, fibroblast, thymic epithelial cell, thyroid epithelial cell, glial cell, pancreatic beta cell, and a vascular endothelial cell. In some embodiments, the APC is a professional APC such as a dendritic cell, macrophage, B cell, or an activated epithelial cell. In other embodiments, the APC is a non-professional APC such as a fibroblast, thymic epithelial cell, thyroid epithelial cell, glial cell, pancreatic beta cell, or a vascular endothelial cell. Since cancer cells also act as antigen presenting cells, the term “antigen presenting cell” further includes cancer cells.

As used herein, “cancer” refers to one of a group of more than one hundred diseases caused by the uncontrolled growth and spread of abnormal cells that can take the form of solid tumors, lymphomas, and non-solid cancers such as leukemia.

The terms “cell,” “cell line,” and “cell culture” include progeny. It is also understood that all progeny may not be precisely identical in DNA content due to deliberate or inadvertent mutations. Variant progeny that have the same function or biological property, as screened for in the originally transformed cell, are included. The “host cells” used in the present invention generally are prokaryotic or eukaryotic hosts. Further, a “cell population” is defined herein as a group of like cells, such as B cells or dendritic cells, in a closed system. A human body is one type of closed system.

A “composition” is intended to mean a combination of active agent and another compound or composition, inert (for example, a detectable agent or label) or active (such as an adjuvant).

As used herein, the term “comprising” is intended to mean that the compositions and methods include the recited elements, but not excluding others. “Consisting essentially of,” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purpose. A control can be “positive” or “negative.” As used herein, a “control polypeptide” is a polypeptide expressed/presented with an MHC class II molecule on the surface of a control-treated APC or an untreated APC.

An “effective amount” is an amount sufficient to effect beneficial or desired results. An effective amount can be administered in one or more administrations, applications or dosages.

The term “epitope” is defined herein as a polypeptide that binds to, is expressed with, and/or is presented at the cell surface with an MHC molecule. Increasing the epitope density of a particular APC means to increase the number or kind of polypeptides bound to, expressed with, and/or presented with an MHC molecule at the APC surface.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. When referring to expression of an HDACi-associated polypeptide, “expression” further includes presentation of the polypeptide on the surface of an APC in an MHC Class II binding cleft. “Overexpression” as applied to a gene refers to the overproduction of the mRNA transcribed from the gene or the protein product encoded by the gene at a level that is 2.5 times higher, preferably 5 times higher, more preferably 10 times higher, than the expression level detected in a control sample.

The Fab fragment contains the constant domain of the light chain and the first constant domain (CH1) of the heavy chain. Fab' fragments differ from Fab fragments by the addition of a few residues at the carboxyl terminus of the heavy chain CH1 domain including one or more cysteines from the antibody hinge region. F(ab′) fragments are produced by cleavage of the disulfide bond at the hinge cysteines of the F(ab′)₂ pepsin digestion product. Additional chemical couplings of antibody fragments are known to those of ordinary skill in the art.

A “gene” refers to a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art.

A “gene product” refers to the amino acid (e.g., peptide or polypeptide) generated when a gene is transcribed and translated.

The term “HDACi-associated polypeptide” refers to a polypeptide that is presented by, or bound to, an MHC molecule on the surface of an APC in an increased amount upon administration of an HDACi compound to the APC as compared to administration of a control compound to the APC. In some embodiments, the HDACi-associated polypeptide expression/presentation at the surface is increased by approximately 1 to 50-fold, 25 to 50-fold, 1 to 25-fold, 10 to 25-fold, 1 to 10-fold, 5 to 10-fold, 1 to 5-fold, or 1 to 2-fold. Examples of HDACi-associated polypeptides are found at the top of Table 1 under the heading of “Entinostat.”

The term “isolated” means separated from constituents, cellular and otherwise, in which the polynucleotide, peptide, polypeptide, protein, antibody, or fragments thereof are normally associated with in nature. In one aspect of this invention, an isolated polypeptide is separated from an MHC molecule with which it is associated in nature. As is apparent to those of skill in the art, a non-naturally occurring polynucleotide, peptide, polypeptide, protein, or antibody, or fragments thereof, does not require “isolation” to distinguish it from its naturally occurring counterpart. In addition, a “concentrated,” “separated,” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, or antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

“Mammal” for purposes of treatment refers to any animal classified as a mammal, including a human, domestic and farm animals, nonhuman primates, and zoo, sports, or pet animals, such as dogs, horses, cats, cows, etc.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single target site. Furthermore, in contrast to conventional (polyclonal) antibody preparations, which typically include different antibodies directed against different determinants (epitopes), each monoclonal antibody is directed against a single determinant on the target. In addition to their specificity, monoclonal antibodies are advantageous in that they may be synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies for use with the present invention may be isolated from phage antibody libraries using the well-known techniques. The parent monoclonal antibodies to be used in accordance with the present invention may be made by the hybridoma method first described by Köhler & Milstein ((1975) Continuous cultures of fused cells secreting antibody of predefined specificity. Nature, 256(5517), 495-497) or may be made by recombinant methods.

The term “parenteral” includes subcutaneous, intravenous, intramuscular, intra-articular, intra-synovial, intrasternal, intrathecal, intrahepatic, intralesional, and intracranial injections or infusion techniques.

The term “particulate” refers to powders, granular substances and the like.

The term “peptidome” is defined herein as a group of peptides presented by, or expressed with, a major histocompatibility complex (MHC) molecule on the surface of an APC under a given set of conditions. The term “peptidome modulation” refers to a change in the kind or number of polypeptides presented/expressed with an MHC molecule at the surface of an APC. Peptidome modulation includes increasing the amount of one or more polypeptides, decreasing the amount of one or more polypeptides, removing one or more polypeptides, and/or adding one or more polypeptides. Modulation of a peptidome results in a change in the epitope density of a particular APC or APC cell population. In one embodiment, the amount of a Cathepsin L1 sensitive peptide in the peptidome is reduced. When modulating a peptidome of an APC cell population, the change in the kind or number of polypeptides presented/expressed with an MHC molecule occurs at the level of the cell population and may not occur in each individual APC within the population. A change at the cell population level occurs when approximately 10%, 25%, 50%, 75%, or 90% of the kind or number of polypeptides presented/expressed with an MHC molecule at the surface of an APC are changed within the APC population.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, emulsions, such as an oil/water or water/oil emulsion, and various types of wetting agents. The compositions also can include stabilizers and preservatives. For examples of carriers, stabilizers and adjuvants, see Martin (Remington's Pharm. Sci., 15th Ed., Mack Publ. Co., Easton, Pa. (1975)).

The term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. Pharmaceutically acceptable carriers that may be used in these compositions include ion exchangers; alumina; aluminum stearate; lecithin; serum proteins, such as human serum albumin; buffer substances, such as phosphates; glycine; sorbic acid; potassium sorbate; partial glyceride mixtures of saturated vegetable fatty acids; water; salts or electrolytes, such as protamine sulfate, disodium hydrogen phosphate, potassium hydrogen phosphate, sodium chloride, or zinc salts; colloidal silica; magnesium trisilicate; polyvinyl pyrrolidone; cellulose-based substances; polyethylene glycol; sodium carboxymethylcellulose; polyacrylates; waxes; polyethylene-polyoxypropylene-block polymers; polyethylene glycol; and wool fat.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The compositions can additionally include lubricating agents such as talc, magnesium stearate, and mineral oil; wetting agents; emulsifying agents; suspending agents; preserving agents such as methyl-, ethyl-, and propyl-hydroxy-benzoates; pH-adjusting agents such as inorganic and organic acids and bases; sweetening agents; and flavoring agents.

The term “pharmaceutically acceptable salts” refers to any acid or base addition salt whose counter-ions are non-toxic to the subject to which they are administered in pharmaceutical doses of the salts. A host of pharmaceutically acceptable salts are well-known in the pharmaceutical field. If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

The terms “pharmaceutically effective amount,” “therapeutically effective amount,” or “therapeutically effective dose” refer to the amount of a compound such as a HDACi that will elicit the biological or medical response of a tissue, system, animal, or human that is being sought by the researcher, veterinarian, medical doctor or other clinician. More particularly, “pharmaceutically effective amount,” “therapeutically effective amount,” or “therapeutically effective dose” refer to the amount of an HDACi that will modulate a peptidome of an APC or APC cell population in a tissue, system, animal, or human that is being sought by the researcher, veterinarian, medical doctor or other clinician.

The term “polypeptide” is used in its broadest sense to refer to a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D or L optical isomers, and amino acid analogs and peptidomimetics. In some embodiments, the polypeptide is 5 to 30 amino acid residues in length. In other embodiments, the polypeptide is 9 to 25 or 15 to 24 amino acid residues in length. Accordingly the present invention includes polypeptides of 5, 6, 7, 8, 9, 10, 11, 12, 13, 14, 15, 16, 17, 18, 19, 20, 21, 22, 23, 24, and 25 amino acid residues in length.

The terms “prevent,” “preventing,” “prevention,” and grammatical variations thereof as used herein refer to a method of partially or completely delaying or precluding the onset or recurrence of a disorder or conditions and/or one or more of its attendant symptoms; barring a subject from acquiring or reacquiring a disorder or condition; or reducing a subject's risk of acquiring or reacquiring a disorder or condition or one or more of its attendant symptoms.

A “subject,” “individual” or “patient,” used interchangeably herein, refers to a vertebrate, preferably a mammal, more preferably a human. Mammals include, but are not limited to, murines, simians, humans, farm animals, sport animals, and pets.

The terms “treat,” “treating,” “treatment,” and grammatical variations thereof, as used herein, include partially or completely delaying, alleviating, mitigating or reducing the intensity of one or more attendant symptoms of a disorder or condition and/or alleviating, mitigating or impeding one or more causes of a disorder or condition as compared with prior to treatment of the subject or as compared with the incidence of such symptom in a general or study population. Treatments according to the invention may be applied preventively, prophylactically, pallatively or remedially.

Compositions and Methods

As discussed above, it is a surprising discovery of the present invention that HDACi compositions modulate the peptidome of APCs. In fact, provided herein is the first description of a small molecule modulator of epitope density in APCs. Accordingly, provided herein is a method of modulating an APC peptidome comprising identifying an APC or an APC cell population that expresses an HDACi-associated polypeptide and administering an HDACi to the APC. Also provided herein is a method of identifying an HDACi-associated peptide comprising administering an HDACi to an APC, isolating the polypeptide from an APC MHC Class II molecule binding cleft, and identifying the polypeptide. In one embodiment, the polypeptide is identified via sequencing.

HDACi's can be divided into several groups: hydroxamic acids, cyclic tetrapeptides and depsipeptides, benzamides, electrophilic ketones, and aliphatic compounds. The hydroxamic acid HDACi's include SAHA (Vorinostat), PXD101 (Belinostat), LAQ824, and LBH589 (Panobinostat). The benzamide HDACi's include MS-275 (Entinostat), C1994, and MGCD0103 (Mocetinostat). In some embodiments, the HDACi is a hydroxamic acid. In other embodiments, the HDACi is a benzamide. It should be understood that the present invention includes administering two or more different HDACi's to an APC or an APC cell population. These different HDACi's can be from the same grouping (i.e., benzamide) or from different groupings (i.e., benzamide and hydroxamic acid).

As defined herein, an “antigen presenting cell” is a cell that presents or contains a Class II major histocompatibility complex (MHC Class II) at its surface. MHC Class II molecules are heterodimers of two homogenous polypeptides, an α chain and a β chain. The Class II MHC binding cleft is formed by the α₁ and β₁ portions of the α and β chains. The MHC Class II molecules can be encoded by HLA-DR, HLA-DQ, and HLA-DP genes. In the examples below, the MHC Class II molecules (i.e., polypeptides) are expressed in terms of the genes by which they are encoded. Accordingly, the present invention includes an MHC Class II molecule encoded by HLA-DR, HLA-DQ, or HLA-DP genes. In one embodiment, an MHC Class molecule is encoded by HLA-DR genes including, but not limited to, HLA-DRA, HLA-DRB1, HLA-DRB3, HLA-DRB4, and HLA-DRB5.

The antigen presenting cell expressing MHC Class II on its surface can be selected from a B cell, dendritic cell, macrophage, activated epithelial cell, fibroblast, thymic epithelial cell, thyroid epithelial cell, glial cell, pancreatic beta cell, and a vascular endothelial cell. In some embodiments, the APC is a professional APC such as a dendritic cell, macrophage, B cell, or an activated epithelial cell. In other embodiments, the APC is a non-professional APC such as a fibroblast, thymic epithelial cell, thyroid epithelial cell, glial cell, pancreatic beta cell, or vascular endothelial cell. Since cancer cells also express Class II MHC, the present invention includes embodiments wherein the APC is a cancer cell.

One or more HDACi's can be administered to an APC or APC cell population via any method known to those of ordinary skill in the art. In some embodiments, the administration is performed in vitro. In other embodiments, the administration is performed in vivo. In still other embodiments, the administration is performed ex vivo. The compounds of the present invention can be administered as frequently as necessary, including hourly, daily, weekly or monthly. The compounds utilized in the pharmaceutical method of the invention are administered at the initial dosage of about 0.0001 mg/kg to about 1000 mg/kg daily. A daily dose range of about 0.01 mg/kg to about 500 mg/kg, or about 0.1 mg/kg to about 200 mg/kg, or about 1 mg/kg to about 100 mg/kg, or about 10 mg/kg to about 50 mg/kg, can be used. The dosages, however, may be varied depending upon the requirements of the patient, the severity of the condition being treated, and the compound being employed. For example, dosages can be empirically determined considering the type and stage of disease diagnosed in a particular patient. The dose administered to a patient in the context of the present invention should be sufficient to affect a beneficial therapeutic response in the patient over time. The size of the dose also will be determined by the existence, nature, and extent of any adverse side-effects that accompany the administration of a particular compound in a particular patient. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. For convenience, the total daily dosage may be divided and administered in portions during the day, if desired. Doses can be given daily, or on alternate days, as determined by the treating physician. Doses can also be given on a regular or continuous basis over longer periods of time (weeks, months or years), such as through the use of a subdermal capsule, sachet or depot or via a patch.

As demonstrated in the Examples below, administration of an HDACi such as SAHA (Vorinostat) or MS-275 (Entinostat) to an APC results in the modulation of the APC peptidome. Both such treatments caused a reduction in the amount of CLIP found associated with the MHC Class II molecules on the surface of the APC. CLIP is the default self-peptide occupying the antigenic peptide groove of HLA-DR when antigenic peptide is low or non-existent. Accordingly, displacement of CLIP indicates that the peptidome has changed by increasing the kind or amount of polypeptide presented on the MHC Class II molecules. Table 1 further demonstrates that the APC peptidome differs between an APC treated with Entinostat and a control. Each of the treated and control APC peptidomes includes unique polypeptides not found in the other APC. At least with regard to Entinostat, or benzamide HDACi's, such peptidome change is associated with an induction of cathepsin L1 mRNA.

These new/unique HDACi-associated polypeptides can be identified by methods known to those of ordinary skill in the art. Following administration of an HDACi to an APC for a given period of time, each HDACi-associated polypeptide can be isolated from the MHC Class II binding cleft and sequenced. These methods are described more specifically in the Examples below. Accordingly, provided herein is a method of identifying an HDACi-associated peptide comprising administering an HDACi to an APC, isolating the polypeptide from an APC MHC Class II molecule binding cleft, identifying the polypeptide, and determining that the polypeptide is differentially expressed (i.e., differentially bound to Class II MHC) on a control APC. In one embodiment, the polypeptide is identified by amino acid sequencing.

These HDACi-associated polypeptides represent a previously untapped group of antigenic epitopes that are then investigated and developed as vaccine candidates and general immune-stimulants/adjuvants. These methods are especially important for personalized medicine approaches because MHC class II proteins are highly polymorphic, i.e., they vary extensively from person to person. Since MHC Class II molecules in one person differ from another person, the immune response to a particular pathogen can also differ. The present invention solves this problem by providing a convenient way to isolate a personal peptide repertoire (HDACi-associated polypeptides) that could be used for stimulating an immune response optimized for each individual.

In some embodiments, the APC is a tumor cell and the HDACi-associated polypeptide is a tumor antigen. Very few MHC Class II tumor antigens have been identified, and the compositions and methods provided herein offer an invaluable opportunity to increase the number of these tumor antigens. Understanding the range of tumor antigens, i.e., peptides that can occupy the HLA-DR binding cleft, will answer many questions related to tumor immunology.

It should be understood that peptidome modulation includes increasing the amount of one or more polypeptides, decreasing the amount of one or more polypeptides, removing one or more polypeptides, and/or adding one or more polypeptides to the group of polypeptides expressed by an APC under control conditions. Modulation of a peptidome results in a change in the epitope density of a particular APC or APC cell population. In one embodiment, the amount of a Cathepsin L1 sensitive peptide is reduced.

When modulating a peptidome of an APC cell population, the change in the kind or number of polypeptides presented/expressed with an MHC molecule occurs at the level of the cell population and may not occur in each individual APC within the population. A change at the cell population level occurs when approximately 10%, 25%, 50%, 75%, or 90% of the kind or number of polypeptides presented/expressed with an MHC molecule at the surface of an APC are changed within the APC population.

It should be understood that the foregoing relates to preferred embodiments of the present disclosure and that numerous changes may be made therein without departing from the scope of the disclosure. The disclosure is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present disclosure and/or the scope of the appended claims. All patents, patent applications, and publications referenced herein are incorporated by reference in their entirety for all purposes.

### EXAMPLES

### Example 1

### HDACi Treatment of Melanoma and B Cells Reduces CLIP Occupancy of MHC Class II

To determine whether HDACi's could affect anti-tumor immunity, major histocompatibility (MHC) class II positive melanoma cells were treated with HDACi's and assayed for potential alterations in the binding of MHC class II to antigenic peptides. More specifically, melanoma cells were treated with Entinostat or Vorinostat, two HDACi's currently used to treat patients. The CIITA-4 melanoma cells were previously transformed with an expression vector for the MHC class II transactivator, CIITA, which leads to constitutive MHC class II expression. The WM9 melanoma cells have naturally occurring CIITA and MHC class II expression. Raji B cells also constitutively express both CIITA and MHC class II.

Cells were cultured in RPMI (antibiotic supplements and 10% fetal calf serum) and treated with either DMSO, 5 μM Entinostat, 3 μM Vorinostat, or 400 units/ml IFN-γ. After 40 hours of treatment, cells were recovered and stained with antibodies for flow cytometry. Surface expression of HLA-DR, the most significant MHC class II protein, was detected using flow cytometry. Surface expression of the class II associated invariant chain peptide (CLIP) was also detected using flow cytometry. CLIP is the default self-peptide occupying the antigenic peptide groove of HLA-DR when antigenic peptide is low or non-existent.

Samples were analyzed on a BD LSR II flow cytometer (San Jose, Calif.) equipped with 405 nm, 488 nm and 633 nm lasers using BD FacsDiva 6.1.3 software. FITC, PE and DAPI dyes were detected using 530/30, 575/26 and 450/50 band pass filters, respectively. Ratios are expressed as the ratio of mean fluorescence index (MFI) differentials, with DMSO treatment normalized to one. The MFI values of PE-labeled isotype control were subtracted from PE-labeled anti-HLA-DR. The MFI values of FITC-labeled isotype control were subtracted from FITC-labeled anti-CLIP. (All antibodies were purchased from BD Pharmigen.) The MFI differentials for CLIP and HLA-DR were converted to CLIP/HLA-DR.

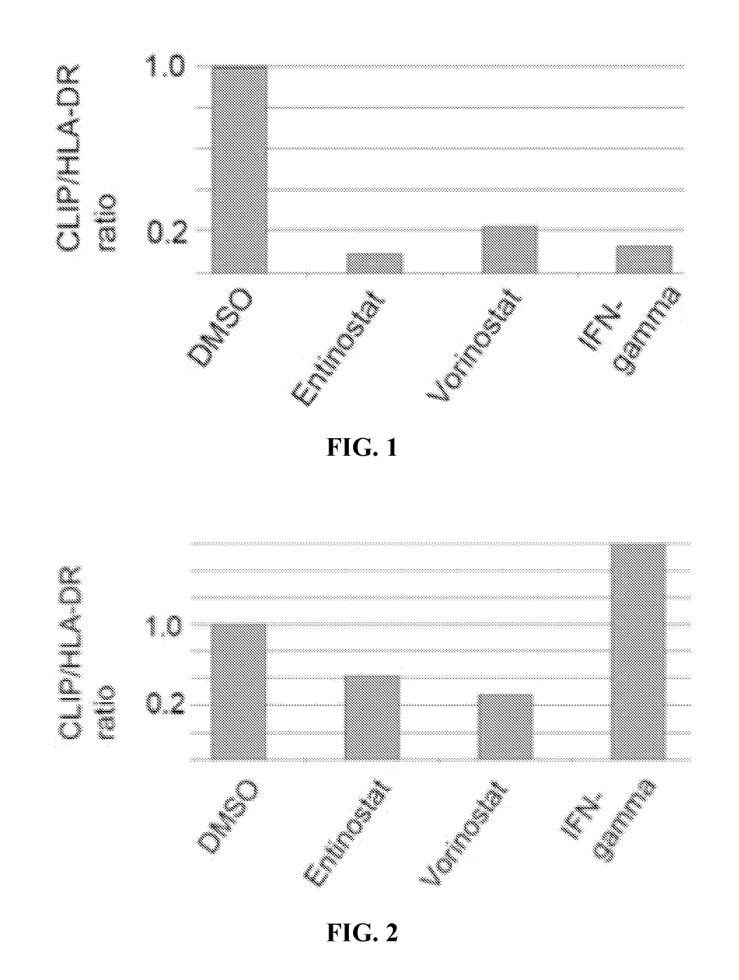

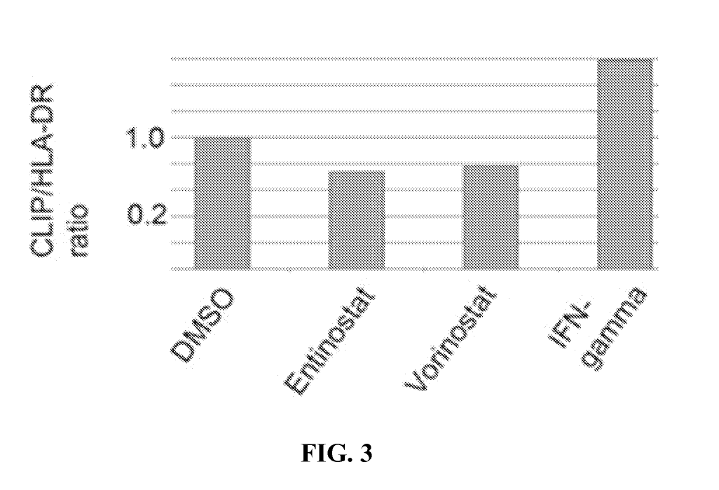

The results obtained indicate that the HDACi's increased HLA-DR surface expression to a modest extent, at best, in cells constitutively expressing HLA-DR. However, CLIP occupancy was substantially reduced in the cells treated with the HDACi's. FIGS. 1-3 show the ratio of CLIP to HLA-DR on the cell surface. A decrease in CLIP occupancy in the groove of the HLA-DR surface protein indicates that CLIP is being replaced by other cellular peptides.

### Example 2

### Entinostat Induces Cathepsin L1 mRNA in WM9 Melanoma and Raji B-Cells

An RT-PCR assay of cathepsin L1 mRNA was performed in treated Raji B-cells. More specifically, RNA was extracted from the DMSO, Entinostat, Vorinostat, and IFN-γ treated Raji B-cells described in Example 1 using an RNeasy Mini Kit< Qiagen cat #74106. RNA quality was verified using a bioanalyzer (Agilent). Reverse transcription of total RNA (1 μg) to cDNA was done with a High Capacity RNA-to-cDNA Master Mix (Applied Biosystems part of Life Technologies, Foster City, Calif., catalog number 4390777). In a 0.2 ml PCR tube, 5×Complete Master Mix (4.0 μl) and nuclease-free H₂0 were mixed with 1 μg RNA for a total reaction volume of 20 μL. The sample was incubated at 25° C. for 5 minutes, 42° C. for 30 minutes and then 85° C. for 5 minutes in a DNA Engine Peltier thermal cycler (Bio-Rad). qPCR target related: cathepsin L1, CTSL1, RefSeq-NM₁₃ 001912.4, amplicon length-85 bp, Assay ID Details-Hs00377632_ml; Cathepsin S, CTSS, RefSeq-NM_{—}004079.4, amplicon length-70 bp, Assay ID Details Hs00175407_ml; GAPDH-glyceraldehyde-3-phosphate dehydrogenase, Gapdh, RefSeq-NM_{—}00246.3, amplicon length-58 bp, Assay ID Details-Hs03929097_gl.

All primers and probes were from Applied Biosystems Assay-on-Demand. Probe was FAM-NFQ. cDNA was diluted in nuclease free water (Ambion, part of Life Technologies, Foster City, Calif., catalog number AM9937) as follows: Template for target CTSL1 at 2-fold, template for target CTSS1 (data not shown) at 10-fold; and template for target GAPDH at 10-fold. Amplification for each target was done in triplicate for each sample and a no-template control was completed for each assay mix. The GAPDH was used as an endogenous control for relative qPCR quantification. Amplification of cDNA that was diluted with water was done with 2×Gene Expression Master Mix (Applied Biosystems part of Life Technologies, Foster City, Calif. catalog number 4369016). In each well of an Optical 96-well Fast Thermal Cycling Plate (Applied Biosystems catalog number 4346906) was a 10 μl reaction containing 2×Gene Expression Master Mix (5 μL), 20×TaqMan Assay-on-Demand primer and probe mix (0.5 μL), diluted cDNA (2.0 μl) and nuclease-free H₂0 (2.5 μl). The plate was sealed with MicroAmp Optical Adhesive Film (Applied Biosystems catalog number 4313663) The sample was incubated at 50° C. for 20 minutes, 95° C. for 10 minutes, then 40 cycles at 95° C. for 15 seconds and 60° C. for 1 minute in a 7500FAST Real-time PCR thermocycler (Applied Biosystems). The qPCR analysis program is 7500 Software v2.0.5 from Applied Biosystems. ΔΔCq determination was used. All no-template controls were negative.

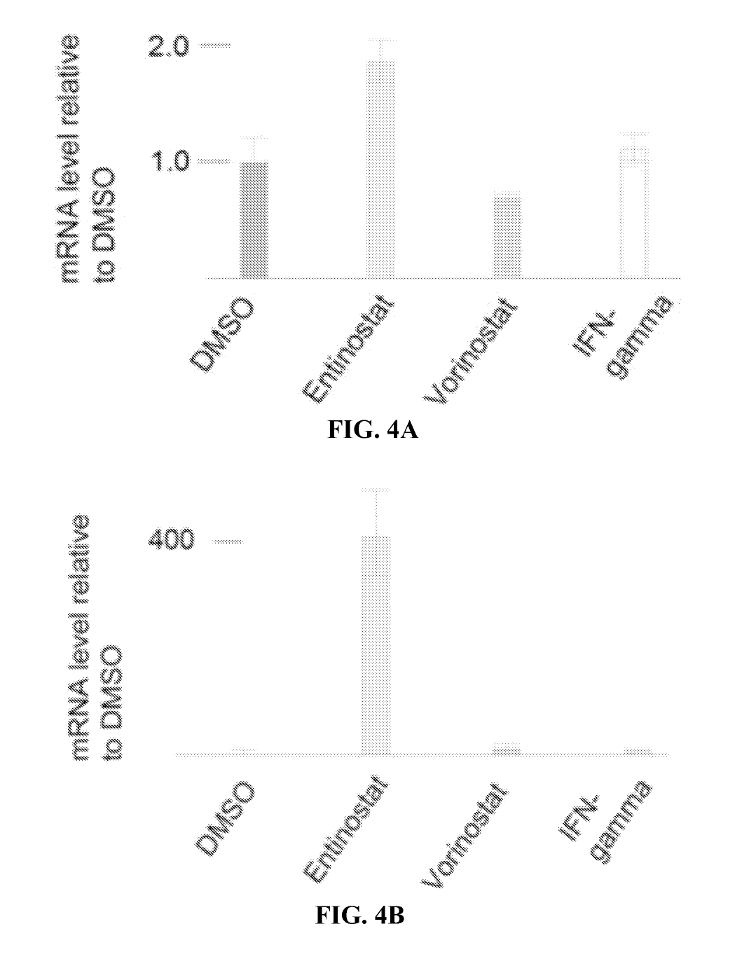

The results of the RT-PCR assay are shown in FIGS. 4A-B (4A relates to WM9 melanoma cells and 4B relates to Raji B cells). It should be noted that Cathepsin L1 RNA levels were normalized to GAPDH mRNA amounts. Results were also normalized to DMSO treatment of cells (given an arbitrary value of 1.0) after normalizing the results to GAPDH mRNA amounts. These results indicate that MS-275 induces cathepsin L1 mRNA in Raji B-cells and in WM9 melanoma cells; no effect was seen for cathepsin S; and no effect was seen for SAHA for either cathepsin L1 or S mRNA induction.

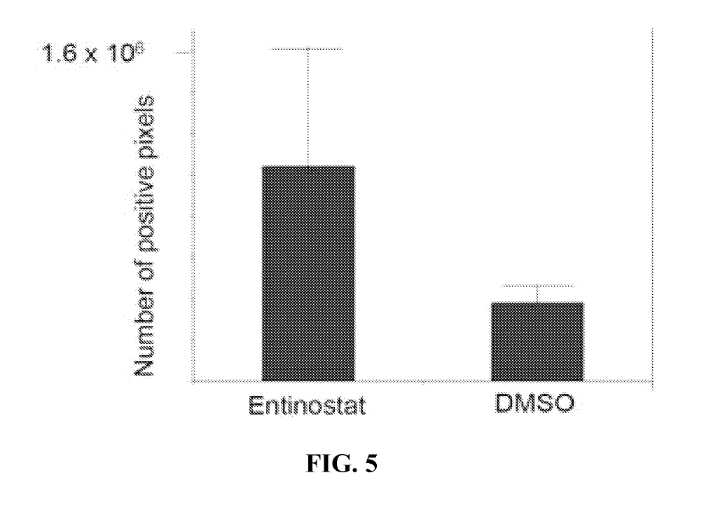

FIG. 5 also demonstrates significant induction of Cathepsin L1 in cells treated with 5 μM Entinostat. In FIG. 6, samples were viewed with a Leica DMLB upright bright field microscope and a 20×/0.5NA HC PL fluortar objective (Leica Microsystems GmbH, Wetzlar, Germany). Transmitted bright light was used without contrasting methods. Gain, offset, and other acquisition settings were identical for all samples within each group. The number of nuclei was determined with auto-threshold segmentation on the cell morphology. The nuclei segmentation was further refined by size and shape of the objects created from the initial segmentation. ImagePro Plus v6.1 (Media Cybernetics, Bethesda, Md.) software was then used to determine the ctsl1 staining intensity (ctsl1 is cathepsin L1 antibody). Positive cells were established as >20DR over background for each 8bit image (0-255DR). The number of pixels over this threshold per cell was used to determine the differences between the Entinostat and DMSO groups. Each image and analysis was blinded and completed in triplicate (p-value <0.0004).

### Example 3

### Modulation of Raji B Cell Peptidome Using Entinostat

All peptide sequences were manually validated for accuracy and specificity (Escobar et al. 2011. Utility of characteristic QTOF MS/MS fragmentation for MHC class I peptides. J. Proteome. Res., 10:2494-2507). To avoid false negative identification of peptides eluted from each condition, the list of possible sequences was inspected to make sure that unique peptides eluted from Entinostat treated (5 μM for 40 hours) cells were absent in DMSO (control) treated cells and vice versa.

In Table 1, P3-P1 represents amino acids (AA) to the left (amino-terminal) side of the cleavage, not in the peptide. P1′-P3′ represents AA to the right side of cleavage, not in the peptide. For the HLA-DR binding peptide, P1′-P3′ are the left-most 3 AA; P3-P1, the right-most 3 AA. AA that are underlined are cathepsin L1 specific. AA that are in large font represent AA favored by both human Cathepsin L1 and S; AA that are double underlined are cathepsin S specific. There are no significant differences between the two groups in the signature AAs associated with the P3-P3′ segments of the peptides. To identify the signature AA's, data and algorithms developed previously were relied upon which employed the approach of proteomic identification of protease cleavage sites, based on proteome derived peptide libraries. (Biniossek, M. L., D. K. Nagler, C. Becker-Pauly, and O. Schilling. 2011. Proteomic identification of protease cleavage sites characterizes prime and non-prime specificity of cysteine cathepsins B, L, and S. J. Proteome. Res. 10:5363-5373). This approach takes into consideration the subsite cooperatively in the amino acid sequence surrounding the cleaved, peptide bond. It was also noted that all Cathepsin B specific AA in both sets were NOT specifically associated with either set.

TABLE 1
Peptides Unique to Entinostat and DMSO (control) treated cells.
Entinostat

| | | HLA-DR Binding Peptide | | | |
|---|---|---|---|---|---|
| Protein | P3-P1 | P1′-P3′ | P3-P1 | P1′-P3′ | Comments |

| | | | | |
|---|---|---|---|---|
| 40S ribosomal protein S16 | ..M | 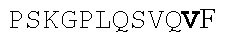 | GRK | ribosomal protein, component of the 40S subunit.; multiple processed |
| | | | | pseudogenes in genome |
| ANKRD26-like family C member 1A | TTM | 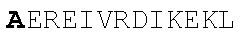 | CYV | prostate, ovary, testis-expressed protein on chromosome 2 |
| C-C chemokine receptor type 7 | ARN | 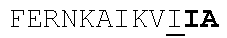 | VVV | G protein-coupled receptor family induced by EBV infection; editor of |
| precursor | | | | EBV effects on B lymphocytes; |
| | | | | expressed in various lymphoid tissues; |
| | | | | activates B and T lymphocytes; |
| | | | | controls migration of memory T cells |
| | | | | to inflamed tissues; stimulates |
| | | | | dendritic cell maturation. |
| CXC motif chemokine 10 precursor | PVN QPV ISN ISN | 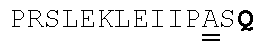 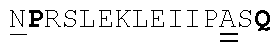 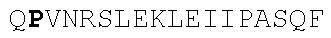 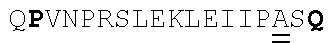 | FCP FCP CPR FCP | chemokine of CXC subfamily; binding of protein to receptor CXCR3 results in numerous effects related to immune activation |
| Heat shock 70 kDa protein 1L | GRD | 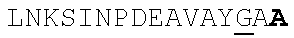 | VQA | stabilizes proteins against aggregation and mediates the folding of newly |
| | | | | translated proteins. |
| Proactivator polypeptide precursor | DSY | 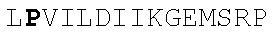 | GEV | |
| Solute carrier family member 23 member 2 | LFQ SA F | 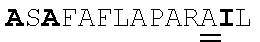 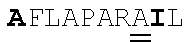 | SLD SLD | one of two required transporters for tissue-specific uptake of vitamin C. |

DMSO

| | | | | |
|---|---|---|---|---|
| Glycerol-3-phosphate transporter | APF | 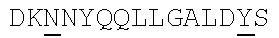 | FLC | transports glyerol-3-phosphate between cellular compartments |
| Hepatitis B virus X- interacting protein | LSD | 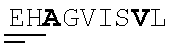 | AQQ | complexes with c-terminus of hepatitis B virus X protein; negatively regulates |
| | | | | HBx activity |
| HLA class II histocompatibility | QGG | 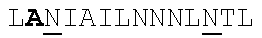 | IQR | alpha chain HLA class II polypeptide |
| antigen, DP alpha | | | | |
| chain precursor | | | | |
| HLA class II histocompatibility antigen, DP(W4) beta | RFD FDS | 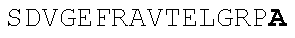 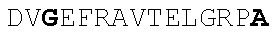 | AEY AEY | beta chain HLA class II polypeptide. |
| chain precursor | | | | |
| Interleukin-4 receptor | EAG | 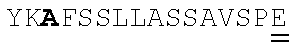 | KCG | transmembrane protein that binds IL4 |
| alpha chain precursor | GEA | 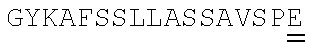 | KCG | and IL13 to regulate IgE production, promote differentiation of Th2 cells. |
| Myosin-9 | NMD | 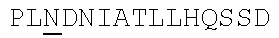 | KFV | non-muscle myosin; involved in cytokinesis, cell motility, and |
| | | | | maintenance of cell shape. |
| Myosin-Ie | QKQ | 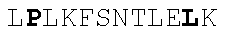 | LKK | non-muscle, class I myosins, a |
| | KQL | 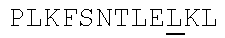 | KKE | subgroup of the nonconventional myosin protein family |
| NEDD4 family- | AGD | 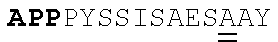 | FDY | thought to be part of a family of |
| interacting protein 1 | AGD | 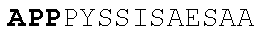 | YFD | integral Golgi membrane proteins |
| Phosphoglycerate kinase 1 | IGG | 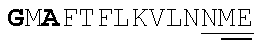 | IGT | catalyzes conversion of 1,3- diphoshpglycerate |
| Serine/threonine- protein kinase 31 | YSL | 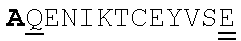 | GNI | putative protein kinase with a tudor domain |
| Sortilin-related receptor precursor | KAH | 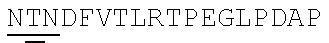 | RNL | likely plays a role in endocytosis; may be associated with Alzheimer's Disease |
| Sterol regulatory element-binding | GAP | 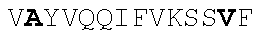 | PWH | |
| protein cleavage | | | | |
| protein | | | | |
| Syntaxin-7 | DKY | 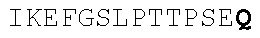 | RQR | possibly involved in protein trafficking from the PM to the endosome; |
| | | | | mediates trafficking to endosomes and |
| | | | | lysosomes. |

### Example 4

### Entinostat Treatment of Raji B Cells Decreases Cathepsin L Sites Within the Peptidome

An initial analysis of the Entinostat dependent peptides in Table 1 does not significantly reflect peptide generation by Entinostat induced cathepsin L1. Both FIG. 4B and FIG. 5 show significant induction of cathepsin L1. Thus, an alternative hypothesis was considered: cathepsin L1 induction favors peptides that DO NOT have cathepsin L1 sites. Using this alternative hypothesis, another algorithm was employed (rather than that used in Table 1), to obtain identification of ranked, cathepsin L1 cleavage sites WITHIN the peptides of Table 1. (Only the largest peptide from each protein represented in Table 1 was analyzed.)

The results are provided in Table 2 below where the shading scheme for signature AA for cathepsins is the same as Table 1. Shading represents the P1′-P3′ AA for the indicated sites. The Cathepsin L1 cleavage site scores and site specificities were obtained from SitePrediction (www.dmbr.ugent.be/prx/bioit2-public/SitePrediction/). In all cases, scores represent the site within the peptide with the maximum score. Table 2 analysis indicated, with a p-value of <0.03, that peptides from DMSO treated cells had higher quality cathepsin L1 sites WITHIN the peptides than did the peptides from the Entinostat treated cells. Interestingly, of the seven distinct proteins represented by the seven peptides at the top of Table 2 (Entinostat), five have relatively high quality cathepsin L1 sites within 10 AA of either side of the peptide bound to HLA-DR.

TABLE 2
Putative Cathepsin L sites within HLA-DR binding peptides

| | | |
|---|---|---|
| Peptide | Cleavage site score | Site specificity |

Entinostat

| | | |
|---|---|---|
| 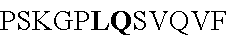 | 190 | >95% |
| 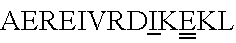 | 98 | >95% |
| 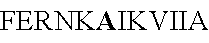 | 11 | <95% |
| 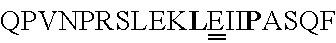 | 1792 | >99% |
| 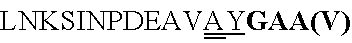 | 17 | >95% |
| 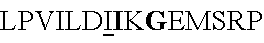 | 21 | >95% |
| 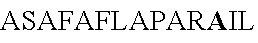 | 9 | >95% |
| Average score | 67.2 | 16/13 |

DMSO

| | | |
|---|---|---|
| 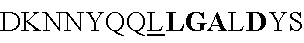 | 521 | >99% |
| 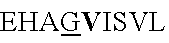 | 10 | <95% |
| 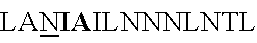 | 0.2 | <95% |
| 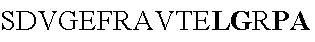 | 579 | >99% |
| 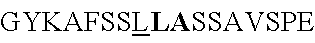 | 343 | >99% |
| 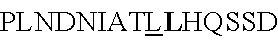 | 383 | >99% |
| 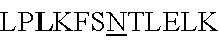 | 62 | >95% |
| 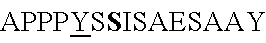 | 223 | >99% |
| 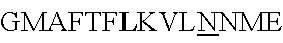 | 109 | >95% |
| 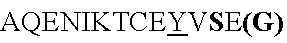 | 4 | <95% |
| 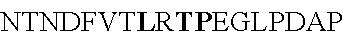 | 23 | <95% |
| 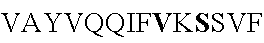 | 147 | >95% |
| 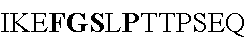 | 455 | >99% |
| Average score | 207.2 | |
| p-value for average scores | <0.03 | 36/27 |

1. A method of identifying an HDACi-associated polypeptide comprising administering an HDACi to an antigen presenting cell (APC), isolating the polypeptide from an APC major histocompatibility complex (MHC) Class II molecule binding cleft, and identifying the polypeptide, wherein expression of the HDACi-associated polypeptide is increased as compared to a control.

2. The method of claim 1, wherein the APC is selected from the group consisting of a B cell dendritic cell, macrophage, activated epithelial cell, fibroblast, thymic epithelial cell, thyroid epithelial cell, glial cell, pancreatic beta cell, and vascular endothelial cell.

3. The method of claim 1, wherein the APC is selected from the group consisting of a B cell dendritic cell, macrophage, and activated epithelial cell.

4. The method of claim 1, wherein the APC is a B cell.

5. The method of claim 1, wherein the APC is a cancer cell.

6. The method of claim 1, wherein the MHC Class II molecule is encoded by HLA-DR genes.

7. The method of claim 1, wherein the HDACi is a benzamide.

8. The method of claim 7, wherein the benzamide is MS-275.

9. The method of claim 1, wherein the HDACi is a hydroxamic acid.

10. The method of claim 9, wherein the hydroxamic acid is suberoylanilide hydroxamic acid.

11. A method of modulating an antigen presenting cell (APC) peptidome comprising identifying an APC or an APC cell population that expresses an HDACi-associated peptide and administering an HDACi to the APC.

12. The method of claim 11, wherein the amount of a Cathepsin L1 sensitive peptide is reduced.

13. The method of claim 11, wherein the APC is selected from the group consisting of a B cell dendritic cell, macrophage, activated epithelial cell, fibroblast, thymic epithelial cell, thyroid epithelial cell, glial cell, pancreatic beta cell, and vascular endothelial cell.

14. The method of claim 11, wherein the APC is a B cell.

15. The method of claim 11, wherein the APC is a cancer cell.

16. The method of claim 11, wherein the MHC Class II molecule is encoded by HLA-DR genes.

17. The method of claim 11, wherein the HDACi is a benzamide.

18. The method of claim 17, wherein the benzamide is MS-275.

19. The method of claim 11, wherein the HDACi is a hydroxamic acid.

20. The method of claim 19, wherein the hydroxamic acid is suberoylanilide hydroxamic acid.
